(19) United States
(12) Patent Application Publication
(10) Pub. No.: US 2022/0112530 A1
(43) Pub. Date: Apr. 14, 2022

(54) KIND OF METHOD FOR PREPARING L-CITRULLINE BY USING AEROMONAS SP.

(71) Applicant: Zhejiang University of Technology, Hangzhou City (CN)

(72) Inventor: Shijin Wu, Hangzhou (CN)

(21) Appl. No.: 17/070,726

(22) Filed: Oct. 14, 2020

(51) Int. Cl. C12P 13/10; C12N 1/20

(52) U.S. Cl. CPC C12P 13/10; C12N 1/20; C12N 2500/05; C12N 2500/32; C12N 2500/34

(57) ABSTRACT

The present invention discloses a kind of method for preparing L-citrulline by using Aeromonas sp., which is related to the technical field of bioengineering. The said method consists of following steps: fermenting Aeromonas sp. in a culture medium to obtain seed liquid, and the liquid is inoculated into the fermenter at an inoculum size of 1-2% by volume. The initial pH is 3.5, and the pH in the medium is controlled below 4.0 all the time. Then, conducts shaking culture for 15-20 hours to obtain the fermentation broth. The fermentation broth is centrifuged to obtain precipitation solution and supernatant; Use the said supernatant to prepare the substrate solution, and the pH of the substrate solution is controlled at 3-5, and then, add the precipitation solution to react at the temperature of 30-50° C., the reaction time is 5-8 hours. After reaction, the L-citrulline is obtained by the method of concentration and crystallization. The present invention provides a method for preparing L-citrulline with low cost, high L-citrulline content and high purity.

### FIELD OF THE INVENTION

The present invention relates to the technical field of bioengineering, particularly involves a kind of method for preparing L-citrulline by using Aeromonas sp.

### BACKGROUND OF THE INVENTION

In 1914, Taro Uga and others isolated citrulline from watermelon juice for the first time, and then Mitsude Wada recognized that it was a kind of amino acid. The citrulline exists in the seeds of cucurbitaceous plants in a free form. Up to now, people have successfully isolated citrulline H1 from watermelon juice, wild watermelon leaves, walnut kernels, walnut seedlings and seeds. Citrulline is a kind of nonprotein amino acid, and research on citrulline are relatively more done by foreign scientists, especially in Japan, the United States and Europe. In China, the research on citrulline is still in the stage of cognition, and there is no corresponding report on the production method, also researches on its detection method and physiological function are relatively less, only a few of literature reports have been kept. Yue Rui et al. used a dual-wavelength TLC-scanning method to measure the content of citrulline in Radix Trichosanthis. In the study of the induction and cultivation of the mutant strain of Neurospora crassa, it was found out that the citrulline had an obvious growth-promoting effect on the mutant strain. In addition, Zheng Chunfu et al. discovered that increasing the exogenous L-citrulline can significantly raise the production of NO induced by activated macrophages, and then reduces the infection rate of activated macrophages as well as inhibits the proliferation of intracellular Toxoplasma Tachyzoites. Zeng Xiaofeng and others also found out that anti-cyclic citrullinated peptide antibodies play an important role in the diagnosis of rheumatoid arthritis.

Citrulline can improve immune system function, maintain the function of joint movement, balance the healthy blood sugar level, and is rich in antioxidants to absorb harmful free radicals, as well as contributes to keep normal level of cholesterol. It can also relax people's blood vessels, enhance male sexual function, and treat sexual dysfunction, and can maintain healthy lung function as well as improve mental clarity, reduce pressure, and overcome depression, and has function of helping brain nerve cells have capability of storing and recalling information. With the rapid development of studies of biotechnology and genetic engineering, the biochemical products market has continued to increase, with the global annual turnover ups to billions of dollars. Therefore, the preservation of quite a lot of biological products such as enzymes, vaccines, and antibodies is more and more concerned. The function of citrulline to scavenge hydroxyl has been well utilized, and relevant studies have shown that the citrulline has protective effect of anti-hydroxyl to DNA and enzymes. As a kind of antioxidant, citrulline can be used in cosmetics, health foods, food additives and nutritional fortifiers, which also has protective effect on anaerobic substances. Therefore, people pay more and more attention to citrulline, and hope to develop new functional foods and functional drugs based on it.

In the aspect of the physiological function of citrulline, foreign scientists have found out that the citrulline has some significant pharmacological functions by studies, such as scavenging free radicals, which is of great value in the health care of human body; effect of vasodilation, which is expected to become an effective medicine for treating endotoxin and sepsis. A Japanese scientist has discovered a new kind of citrulline polypeptide and developed this new polypeptide into an anti-AIDS drug. At the same time, the citrulline is identified as a very effective antioxidant, and this function has been used in various fields such as cosmetics, medicines, and health foods. In recent years, quite a lot of overseas studies have shown that the citrulline has many important physiological functions, such as scavenging free radicals, being an indicator of allograft rejection, effect of vasodilation, stabilizing blood pressure, and diagnosing rheumatoid arthritis, as well as antioxidant, etc., and thus has a wide application prospect.

There are three main methods for preparing citrulline. Fermentation and enzymatic methods are mainly used in foreign countries, while such common-used separation methods as ion exchange are mostly used for separation and purification.

(1) Chemical method: refers to hydrolyzing L-arginine under alkaline conditions to obtain L-citrulline, which is hard to control the process and its product contains the optical antipode D-citrulline, so that affects the quality of the product and produces a lot of waste water during the process of production, and thus pollutes the environment. However, the chemical method is the only way for the industrial production of L-citrulline in China.

(2) The difficulty of fermentation production is that the yield of L-citrulline per unit volume is low, the highest only reaches 1.7 g/L, and the cost of extracting L-citrulline from the fermentation broth is relatively high. The advantages of the fermentation method are low cost, high yield, the purity of product is high, and there is no toxic substance in the process of production, and thus provides convenience for the post-processing of the product as well as reduces cost. Nowadays, Japan is the country that conducted most research on the production of citrulline by means of fermentation, which can be traced back to the 1930s and reached a certain level in the 1960s.

(3) Enzymatic method: refers to that the L-arginine is converted into the L-citrulline under the action of arginine deiminase, and the production conditions of which are mild, also there is no toxic substance, and fewer impurities has been produced in the conversion system, in addition, the extraction process is simple with less pollution. This method uses microbial cells with strong specificity and high conversion rate as a catalyst to catalyze the group of arginine deiminase to produce L-citrulline. Using enzymatic method to synthesize citrulline usually applies a one-step enzymatic reaction, and thus can avoid the complex feedback regulation occurred in the complete synthesis pathway of citrulline, as well as allows the citrulline to be accumulated to higher concentrations.

The fermentation method is favored by researchers due to the low-cost raw material, and has made great progress in recent years, which is mainly manifested in the selection and breeding of strains and the reformation of production technology. As early as the 1950s, Japanese researchers have used such traditional induced mutation techniques as ultraviolet and cyanide to obtain the deficient citrulline mutants of corynebacterium glutamicum, and thus successfully accumulated L-ornithine. Wan Honggui et al. disclosed a method for preparing L-ornithine by using Corynebacterium glutamicum CGMCC No. 3663 for fermentation, and the L-ornithine in the fermentation broth was accumulated to 35-45 g/L. However, the fermentation method also has some disadvantages. Such as the instability of strains, the control of fermentation process parameters and other burning problems occurred during the process of separation and extraction of L-ornithine in the fermentation broth.

The process of preparing L-citrulline by enzymatic conversion requires arginine deiminase. Currently, adding a small amount of L-arginine to induce is necessary for the process of preparing arginine deiminase by using wild-type strains for fermentation, and the L-arginine is converted into L-ornithine after catabolizing, which is accumulated in the fermentation broth. Since less L-arginine is added during the induction process, the concentration of L-ornithine accumulated in the fermentation broth is relatively low, and thus the cost of separation and purification is relatively high. The fermentation broth is usually treated as wastewater, which not only pollutes the environment, but also wastes resource. In addition, the cost of preparing L-citrulline by using pure L-arginine as a substrate for enzymatic conversion is relatively high. Therefore, it is urgently needed to provide a method for preparing L-citrulline characterized in low cost, high content of L-citrulline, and high purity.

### SUMMARY OF THE INVENTION

The purpose of the present invention is to provide a method for preparing L-citrulline characterized in low cost, high content of L-citrulline, and high purity to solve the problems existing in the prior art.

In order to achieve the above objective, the present invention provides the following scheme:

The present invention provides a method for preparing L-citrulline by using Aeromonas sp., which consists of following steps:

(1) Cultivate strains: Ferment Aeromonas sp. in the culture medium to obtain seed liquid, and inoculate the seed liquid in a 200-300 L fermenter at an inoculum size of 1-2% by volume. The initial pH is 3.5, and the pH in the medium is controlled below 4.0 all the time, the loaded liquid volume of the fermentation tank is ⅓ of the total volume, the volume of aeration is 12-15 m³/h, the tank pressure is 0.03-0.05 MPa, the rotation speed is 80-90 r/min, and the culture temperature is at 35-37° C. Add L-arginine solution during the process of fermentation, then conducts shaking culture for 15-20 hours, and thus obtains fermentation broth. After that, the fermentation broth is centrifuged, and thus obtains precipitation solution and supernatant;

(2) Prepare L-citrulline: prepare a substrate solution with the said supernatant, and the pH of the substrate solution is controlled at 3-5, add the precipitation solution obtained in step (1), react at the temperature of 30-50° C., and the reaction time is 5-8 hours. After reaction, the L-citrulline feed liquid is obtained, and thus obtains L-citrulline by the method of concentration and crystallization.

As a further improvement of the present invention, the said culture medium consists of following components: glucose 0.5-1.0 g/mL, (NH4)2HPO4 0.02-0.3 g/mL, arginine 0.01-0.03 g/mL, (NH4)2HPO4 0.01-0.08 g/mL, manganese sulfate 0.02-0.07 g/mL, starch hydrolysate 2-5 g/mL, glutamic acid 0.001-0.005 g /mL, aspartic acid 0.01-0.03 g/mL, NaCl0.3-0.6 g/mL.

As a further improvement of the present invention, adding L-arginine solution in step (1) to make the concentration of L-arginine in the fermentation broth be 5-15 g/L.

As a further improvement of the present invention, the concentration of L-arginine in the substrate solution in step (2) is 100-130 g/L.

As a further improvement of the present invention, the said method of obtaining L-citrulline by concentration and crystallization described in step (2) specifically consists of following steps:

a. decolor and concentrate the prepared L-citrulline feed liquid, and thus obtains the first batch of L-citrulline concentrates;

b. crystallize the first batch of the L-citrulline concentrates obtained in step a, and thus obtains the first batch of crude L-citrulline and the feed liquid after the crude L-citrulline is separated;

c. concentrate the feed liquid after the crude L-citrulline is separated in step b, and thus obtains the second batch of L-citrulline concentrates;

d. crystallize the second batch of L-citrulline concentrates obtained in step c, and thus obtains the second batch of crude L-citrulline and the feed liquid after the crude L-citrulline is separated; the said crystallization conditions are the same as those in step b;

e. repeat steps c and d, and thus prepares the third batch of L-citrulline, which is the finished L-citrulline.

As a further improvement of the present invention, the said decolorization described in step a refers to adding activated carbon to the L-citrulline feed liquid, and thus obtains decolorized L-citrulline feed liquid; the amount of activated carbon added is 0.5-0.6 wt % of the mass of the L-citrulline feed liquid.

As a further improvement of the present invention, the said concentration described in step a refers to stirring the said decolorized L-citrulline feed liquid at the temperature of 70-80° C. and under the vacuum condition of −0.08-0.07 MPa, when the Baume degree of the said decolorized L-citrulline feed liquid reaches 1.085-1.095, stop stirring, and thus obtains the L-citrulline concentrates; the said speed of stirring is 70-90 r/min.

As a further improvement of the present invention, the said crystallization described in step b refers to lowering the temperature of the first batch of L-citrulline concentrates to 0-5° C., and stirring the concentrates at a speed of 30-40 r/min in the meantime of cooling down, and thus obtains the crude L-citrulline.

As a further improvement of the present invention, the said concentration described in step c refers to stirring the said L-citrulline concentrates at the temperature of 70-80° C. and under the vacuum condition of −0.08-0.07 MPa, when the Baume degree of the L-citrulline concentrates reaches 1.250-1.325, stop stirring, and thus obtains the second batch of L-citrulline concentrates; the said speed of stirring is 70-90 r/min.

The present invention also provides a kind of L-citrulline prepared by the said preparation method, wherein the optical rotation of the said L-citrulline is 24.8°-25.75°, the light transmittance is >99.4%, the purity is >60% and the recovery is >80%.

The present invention discloses the following technical effects:

The present invention uses Aeromonas sp. to prepare L-citrulline, wherein the L-citrulline with high purity can be obtained under the condition of the pH is 3-5 and at the temperature of 30-50° C. to react for 3-5 hours, and the amount of the L-arginine solution added is small, the reaction time is short, the content of L-citrulline is high, and the by-products are small. The method for preparing L-citrulline of the present invention has the characteristics of mild reaction conditions, simple process flow, and suitability for industrial mass production.

### DETAILED DESCRIPTION OF THE INVENTION

Various exemplary embodiments of the present invention will be described in detail herein. The detailed description should not be constructed as a limitation to the present invention, but should be understood as a more detailed description of certain aspects, characteristics, and embodiments of the present invention.

It should be interpreted that the said terms described in the present invention are only used to describe specific embodiments rather than limiting the present invention. In addition, as for the numerical range described in the present invention, it should be interpreted that each intermediate value between the upper and the lower limits of the range is also specifically disclosed. Each smaller range between any stated value or intermediate value within the stated range and any other stated value or intermediate value within the stated range is also included in the present invention. The upper and lower limits of these smaller ranges can be independently included or excluded from the range.

Unless otherwise specified, all technical and scientific terms used herein have the same meaning as commonly understood by those skilled in the said art of the present invention. Although the present invention only describes preferred methods and materials, any method and material similar or equivalent to those described herein can also be used in the embodiment or testing of the present invention. All documents mentioned in the present specification are incorporated by reference, and used to disclose and describe methods and/or materials related to the said documents. In the event of conflict with any incorporated document, the content of the present specification shall prevail.

Without departing from the scope or spirit of the present invention, various improvements and changes can be made to the specific embodiments of the present specification, which is obvious to those skilled in the art. Other embodiments derived from the specification of the present invention will be obvious to the skilled person. The specification and embodiments of the present invention are only exemplary.

Such terms as “consist of”, “include”, “have”, “contain”, etc., used herein are all open words, which mean including but not limited to.

The said Aeromonas sp. of the present invention is Aeromonas sp. YQ, which is preserved in the Chinese Type Culture Collection, the preservation number: CCTCC No: M 2015271, the preservation date: May 3, 2015, and the address: Wuhan University, Wuhan, China, 430072.

The said bacterial strain of the present invention is obtained from: the soil samples collected from the watermelon greenhouse in Gouzhuang, Hangzhou City, Zhejiang Province, which is subjected to primary screening and secondary screening, and thus obtains a pure culture capable of converting the substrate L-arginine to produce L-citrulline. The Morphological, physiological, and biochemical identification of the pure culture was carried out, and it was identified as Aeromonas sp., named as Aeromonas sp. YQ.

### Embodiment 1

The said method for preparing L-citrulline by using Aeromonas sp. in this embodiment consists of following steps:

(1) Cultivate strains: Ferment Aeromonas sp. in the culture medium to obtain seed liquid, and inoculate the seed liquid in a 300 L fermenter at an inoculum size of 1% by volume. The initial pH is 3.5, and the pH in the medium is controlled below 4.0 all the time, the loaded liquid volume of the fermentation tank is ⅓ of the total volume, the volume of aeration is 12 m³/h, the tank pressure is 0.03 MPa, the rotation speed is 80 r/min, and the culture temperature is at 35° C. Add L-arginine solution during the process of fermentation to make the concentration of L-arginine in the fermentation broth is 5 g/L, then conducts shaking culture for 15 hours, and thus obtains fermentation broth. After that, the fermentation broth is centrifuged, and thus obtains precipitation solution and supernatant;

The said culture medium consists of following components: glucose 0.5 g/mL, (NH4)2HPO4 0.3 g/mL, arginine 0.03 g/mL, (NH4)2HPO4 0.01 g/mL, manganese sulfate 0.07 g/mL, starch hydrolysate 2 g/mL, glutamic acid 0.001 g/mL, aspartic acid 0.03 g/mL, NaCl 0.6 g/mL;

(2) Prepare L-citrulline: prepare a substrate solution with the said supernatant, and the concentration of L-arginine in the substrate solution is 100 g/L, the pH of the substrate solution is controlled at 3, add the precipitation solution obtained in step (1), react at the temperature of 30° C., and the reaction time is 8 hours. After reaction, the L-citrulline feed liquid is obtained, and thus obtains L-citrulline by the method of concentration and crystallization.

The said method of obtaining L-citrulline by concentration and crystallization described in step (2) specifically consists of following steps:

a. add activated carbon to the L-citrulline feed liquid for decolorization and concentration, and thus obtains the first batch of L-citrulline concentrates. The amount of activated carbon added is 0.5 wt % of the mass of the L-citrulline feed liquid, the said concentration refers to stirring the said decolorized L-citrulline feed liquid at the temperature of 70° C. and under the vacuum condition of −0.08 MPa, when the Baume degree of the said decolorized L-citrulline feed liquid reaches 1.085-1.095, stop stirring, and thus obtains the L-citrulline concentrates; the said speed of stirring is 70 r/min;

b. crystallize the first batch of the L-citrulline concentrates obtained in step a, and thus obtains the first batch of crude L-citrulline and the feed liquid after the crude L-citrulline is separated, the said crystallization refers to lowering the temperature of the L-citrulline concentrates to 0° C., and stirring the concentrates at a speed of 30 r/min in the meantime of cooling down, and thus obtains the crude L-citrulline;

c. concentrate the feed liquid after the crude L-citrulline is separated in step b, and thus obtains the second batch of L-citrulline concentrates, the said concentration refers to stirring the said L-citrulline concentrates at the temperature of 70° C. and under the vacuum condition of −0.08 MPa, when the Baume degree of the L-citrulline concentrates reaches 1.250-1.325, stop stirring, and thus obtains the second batch of L-citrulline concentrates; the said speed of stirring is 90 r/min;

d. crystallize the second batch of L-citrulline concentrates obtained in step c, and thus obtains the second batch of crude L-citrulline and the feed liquid after the crude L-citrulline is separated; the said crystallization conditions are the same as those in step b;

e. repeat steps c and d, and thus prepares the third batch of L-citrulline.

The optical rotation of the finished L-citrulline prepared in the present embodiment is 24.8°, the light transmittance is 99.8%, the purity of L-citrulline is 70%, and the total recovery is 80%.

### Embodiment 2

The said method for preparing L-citrulline by using Aeromonas sp. in this embodiment consists of following steps:

(1) Cultivate strains: Ferment Aeromonas sp. in the culture medium to obtain seed liquid, and inoculate the seed liquid in a 200 L fermenter at an inoculum size of 1% by volume. The initial pH is 3.5, and the pH in the medium is controlled below 4.0 all the time, the loaded liquid volume of the fermentation tank is ⅓ of the total volume, the volume of aeration is 15 m³/h, the tank pressure is 0.05 MPa, the rotation speed is 90 r/min, and the culture temperature is at 37° C. Add L-arginine solution during the process of fermentation, and the concentration of L-arginine added in the fermentation broth is 15 g/L, then conducts shaking culture for 20 hours, and thus obtains fermentation broth. After that, the fermentation broth is centrifuged, and thus obtains precipitation solution and supernatant;

The said culture medium consists of following components: glucose 1.0 g/mL, (NH4)2HPO4 0.08 g/mL, arginine 0.01 g/mL, (NH4)2HPO4 0.08 g/mL, manganese sulfate 0.02 g/mL, starch hydrolysate 3 g/mL, glutamic acid 0.003 g /mL, aspartic acid 0.02 g/mL, NaCl 0.5 g/mL;

(2) Prepare L-citrulline: prepare a substrate solution with the said supernatant, and the concentration of L-arginine in the substrate solution is 120 g/L, the pH of the substrate solution is controlled at 4, add the precipitation solution obtained in step (1), react at the temperature of 50° C., and the reaction time is 5 hours. After reaction, the L-citrulline is obtained by the method of concentration and crystallization.

The said method of obtaining L-citrulline by concentration and crystallization described in step (2) specifically consists of following steps:

a. add activated carbon to the L-citrulline feed liquid for decolorization and concentration, and thus obtains the first batch of L-citrulline concentrates. The amount of activated carbon added is 0.6 wt % of the mass of the L-citrulline feed liquid, the said concentration refers to stirring the said decolorized L-citrulline feed liquid at the temperature of 80° C. and under the vacuum condition of 0.07 MPa, when the Baume degree of the said decolorized L-citrulline feed liquid reaches 1.085-1.095, stop stirring, and thus obtains the L-citrulline concentrates; the said speed of stirring is 80 r/min;

b. crystallize the first batch of the L-citrulline concentrates obtained in step a, and thus obtains the first batch of crude L-citrulline and the feed liquid after the crude L-citrulline is separated, the said crystallization refers to lowering the temperature of the L-citrulline concentrates to 3° C., and stirring the concentrates at a speed of 35 r/min in the meantime of cooling down, and thus obtains the crude L-citrulline;

c. concentrate the feed liquid after the crude L-citrulline is separated in step b, and thus obtains the second batch of L-citrulline concentrates, the said concentration refers to stirring the said L-citrulline concentrates at the temperature of 75° C. and under the vacuum condition of 0.07 MPa, when the Baume degree of the L-citrulline concentrates reaches 1.250-1.325, stop stirring, and thus obtains the second batch of L-citrulline concentrates; the said speed of stirring is 80 r/min;

d. crystallize the second batch of L-citrulline concentrates obtained in step c, and thus obtains the second batch of crude L-citrulline and the feed liquid after the crude L-citrulline is separated; the said crystallization conditions are the same as those in step b;

e. repeat steps c and d, and thus prepares the third batch of L-citrulline.

The optical rotation of the finished L-citrulline prepared in the present embodiment is 25.45°, the light transmittance is 99.8%, the purity of L-citrulline is 75%, and the total recovery is 83%.

### Embodiment 3

The said method for preparing L-citrulline by using Aeromonas sp. in this embodiment consists of following steps:

(1) Cultivate strains: Ferment Aeromonas sp. in the culture medium to obtain seed liquid, and inoculate the seed liquid in a 300 L fermenter at an inoculum size of 2% by volume. The initial pH is 3.5, and the pH in the medium is controlled below 4.0 all the time, the loaded liquid volume of the fermentation tank is ⅓ of the total volume, the volume of aeration is 15 m³/h, the tank pressure is 0.04 MPa, the rotation speed is 85 r/min, and the culture temperature is at 36° C. Add L-arginine solution during the process of fermentation, and the concentration of L-arginine added in the fermentation broth is 8 g/L, then conducts shaking culture for 20 hours, and thus obtains fermentation broth. After that, the fermentation broth is centrifuged, and thus obtains precipitation solution and supernatant;

The said culture medium consists of following components: glucose 0.8 g/mL, (NH4)2HPO4 0.08 g/mL, arginine 0.02 g/mL, (NH4)2HPO4 0.05 g/mL, manganese sulfate 0.03 g/mL, starch hydrolysate 3 g/mL, glutamic acid 0.003 g /mL, aspartic acid 0.02 g/mL, NaCl 0.5 g/mL;

(2) Prepare L-citrulline: prepare a substrate solution with the said supernatant, and the concentration of L-arginine in the substrate solution is 130 g/L, the pH of the substrate solution is controlled at 5, add the precipitation solution obtained in step (1), react at the temperature of 30° C., and the reaction time is 8 hours. After reaction, the L-citrulline is obtained by the method of concentration and crystallization.

The said method of obtaining L-citrulline by concentration and crystallization described in step (2) specifically consists of following steps:

a. add activated carbon to the L-citrulline feed liquid for decolorization and concentration, and thus obtains the first batch of L-citrulline concentrates. The amount of activated carbon added is 0.6 wt % of the mass of the L-citrulline feed liquid, the said concentration refers to stirring the said decolorized L-citrulline feed liquid at the temperature of 75° C. and under the vacuum condition of 0.07 MPa, when the Baume degree of the said decolorized L-citrulline feed liquid reaches 1.085-1.095, stop stirring, and thus obtains the L-citrulline concentrates; the said speed of stirring is 90 r/min;

b. crystallize the first batch of the L-citrulline concentrates obtained in step a, and thus obtains the first batch of crude L-citrulline and the feed liquid after the crude L-citrulline is separated, the said crystallization refers to lowering the temperature of the L-citrulline concentrates to 5° C., and stirring the concentrates at a speed of 40 r/min in the meantime of cooling down, and thus obtains the crude L-citrulline;

c. concentrate the feed liquid after the crude L-citrulline is separated in step b, and thus obtains the second batch of L-citrulline concentrates, the said concentration refers to stirring the said L-citrulline concentrates at the temperature of 80° C. and under the vacuum condition of 0.05 MPa, when the Baume degree of the L-citrulline concentrates reaches 1.250-1.325, stop stirring, and thus obtains the second batch of L-citrulline concentrates; the said speed of stirring is 80 r/min;

d. crystallize the second batch of L-citrulline concentrates obtained in step c, and thus obtains the second batch of crude L-citrulline and the feed liquid after the crude L-citrulline is separated; the said crystallization conditions are the same as those in step b;

e. repeat steps c and d, and thus prepares the third batch of L-citrulline.

The optical rotation of the finished L-citrulline prepared in the present embodiment is 25.75°, the light transmittance is 99.9%, the purity of L-citrulline is 73%, and the total recovery is 85%.

### Comparative Example 1

Same as the Embodiment 3, the only difference is that the initial pH in step (1) is 6.8, and the pH is maintained at about 7.2 during the fermentation process.

The optical rotation of the finished L-citrulline prepared in the present comparative example is 23.6°, the light transmittance is 99.1%, the purity of L-citrulline is 33%, and the total recovery is 35%.

### Comparative Example 2

Same as the Embodiment 3, the only difference is that the pH of the substrate solution in step (2) is controlled at 6.

The optical rotation of the finished L-citrulline prepared in the present comparative example is 23.1°, the light transmittance is 99.2%, the purity of L-citrulline is 31%, and the total recovery is 33%.

The above-mentioned embodiments only describe the preferred mode of the present invention, rather than limiting the scope of the present invention. Without departing from the design and the spirit of the present invention, any variation and improvement to the technical schemes of the present invention made by those skilled in the art shall fall within the protection scope determined by the claims of the present invention.

1. A kind of method for preparing L-citrulline by using Aeromonas sp., characterized in that it consists of following steps:
(1) Cultivate strains: Ferment Aeromonas sp. in the culture medium to obtain seed liquid, and inoculate the seed liquid in the fermenter at an inoculum size of 1-2% by volume. The initial pH is 3.5, and the pH in the medium is controlled below 4.0 all the time, the loaded liquid volume of the fermentation tank is ⅓ of the total volume, the volume of aeration is 12-15 m³/h, the tank pressure is 0.03-0.05 MPa, the rotation speed is 80-90 r/min, and the culture temperature is at 35-37° C. Add L-arginine solution during the process of fermentation, then conducts shaking culture for 15-20 hours, and thus obtains fermentation broth. After that, the fermentation broth is centrifuged, and thus obtains precipitation solution and supernatant;
(2) Prepare L-citrulline: prepare a substrate solution with the said supernatant, and the pH of the substrate solution is controlled at 3-5, add the precipitation solution obtained in step (1), react at the temperature of 30-50° C., and the reaction time is 5-8 hours. After reaction, the L-citrulline feed liquid is obtained, and thus obtains L-citrulline by the method of concentration and crystallization.

2. The said method for preparing L-citrulline by using Aeromonas sp. according to claim 1, characterized in that the said culture medium consists of following components: glucose 0.5-1.0 g/mL, (NH4)2HPO4 0.02-0.3 g/mL, arginine 0.01-0.03 g/mL, (NH4)2HPO4 0.01-0.08 g/mL, manganese sulfate 0.02-0.07 g/mL, starch hydrolysate 2-5 g/mL, glutamic acid 0.001-0.005 g/mL, aspartic acid 0.01-0.03 g/mL, NaCl0.3-0.6 g/mL.

3. The said method for preparing L-citrulline by using Aeromonas sp. according to claim 1, characterized in that adding L-arginine solution in step (1) to make the concentration of L-arginine in the fermentation broth be 5-15 g/L.

4. The said method for preparing L-citrulline by using Aeromonas sp. according to claim 1, characterized in that the concentration of L-arginine in the substrate solution in step (2) is 100-130 g/L.

5. The said method for preparing L-citrulline using Aeromonas sp. according to claim 1, characterized in that the said method of obtaining L-citrulline by concentration and crystallization described in step (2) specifically consists of following steps:
a. decolor and concentrate the prepared L-citrulline feed liquid, and thus obtains the first batch of L-citrulline concentrates;
b. crystallize the first batch of the L-citrulline concentrates obtained in step a, and thus obtains the first batch of crude L-citrulline and the feed liquid after the crude L-citrulline is separated;
c. concentrate the feed liquid after the crude L-citrulline is separated in step b, and thus obtains the second batch of L-citrulline concentrates;
d. crystallize the second batch of L-citrulline concentrates obtained in step c, and thus obtains the second batch of crude L-citrulline and the feed liquid after the crude L-citrulline is separated; the said crystallization conditions are the same as those in step b;
e. repeat steps c and d, and thus prepares the third batch of L-citrulline, which is the finished L-citrulline.

6. The said method for preparing L-citrulline by using Aeromonas sp. according to claim 5, characterized in that the said decolorization described in step a refers to adding activated carbon to the L-citrulline feed liquid, and thus obtains decolorized L-citrulline feed liquid; the amount of activated carbon added is 0.5-0.6 wt % of the mass of the L-citrulline feed liquid.

7. The said method for preparing L-citrulline by using Aeromonas sp. according to claim 5, characterized in that, the said concentration described in step a refers to stirring the said decolorized L-citrulline feed liquid at the temperature of 70-80° C. and under the vacuum condition of −0.08-0.07 MPa, when the Baume degree of the said decolorized L-citrulline feed liquid reaches 1.085-1.095, stop stirring, and thus obtains the L-citrulline concentrates; the said speed of stirring is 70-90 r/min.

8. The said method for preparing L-citrulline by using Aeromonas sp. according to claim 5, characterized in that the said crystallization described in step b refers to lowering the temperature of the first batch of L-citrulline concentrates to 0-5° C., and stirring the concentrates at a speed of 30-40 r/min in the meantime of cooling down, and thus obtains the crude L-citrulline.

9. The said method for preparing L-citrulline by using Aeromonas sp. according to claim 5, characterized in that the said concentration described in step c refers to stirring the said L-citrulline concentrates at the temperature of 70-80° C. and under the vacuum condition of −0.08-0.07 MPa, when the Baume degree of the L-citrulline concentrates reaches 1.250-1.325, stop stirring, and thus obtains the second batch of L-citrulline concentrates; the said speed of stirring is 70-90 r/min.

10. A kind of L-citrulline, characterized in that is prepared by any of the preparation methods described in claims 1-9, and the optical rotation of the said L-citrulline is 24.8°-25.75°, the light transmittance is >99.99%.
